(19) United States
(12) Patent Application Publication
(10) Pub. No.: US 2023/0309820 A1
(43) Pub. Date: Oct. 5, 2023

(54) METHOD FOR RESTORING BIFOVEAL FUSION IN THE CASE OF ANISOMOTILITY

(71) Applicant: "MODEMI COMPOSITES AND TECHNOLOGIES" LLC, Yerevan (AM)

(72) Inventors: Igor AZNAURYAN, Yerevan (AM); Viktoriya Olegovna BALASANYAN, Yerevan (AM); Erik Igorevich AZNAURYAN, Watertown, MA (US); Satenik Gagikovna AGAGULYAN, Gomel region (BY)

(73) Assignee: "MODEMI COMPOSITES AND TECHNOLOGIES" LLC, Yerevan (AM)

(21) Appl. No.: 18/021,871

(22) PCT Filed: May 18, 2022
(86) PCT No.: PCT/EA2022/050005

§ 371 Date: Feb. 17, 2023

(30) Foreign Application Priority Data
Mar. 19, 2021 (RU) 2021107237

(51) Int. Cl. A61B 3/08; A61F 9/02; A61B 3/113; A61H 5/00

(52) U.S. Cl. CPC A61B 3/08; A61F 9/02; A61B 3/113; A61H 5/00; A61H 2201/5069

(57) ABSTRACT

The method relates to the field of ophthalmology and is intended for postoperative restoring bifoveal fusion in patients, inter alia in children, following concomitant esotropia surgery, with use of liquid crystal glasses. The technical effect of the invention is restoration of bifoveal fusion in accordance with individual ocular motility parameters, using anharmonical alternating of liquid crystal glasses. The technical effect is achieved by the approach during which for patients with a residual angle of deviation of less than 10 degrees after esotropia surgery, a duration of the fixative eye movement of the eyeball (t in ms) is determined for each eye using a Gazelab video eye tracking device produced by BCN Innova (Spain), a duration of occlusion is then calculated in ms for each eye, using the formula: t=40+U, where t is the duration of occlusion in ms; 40 is the minimum time required for visualization between alternations in ms; U is the duration of the fixative eye movement in ms, and the duration of occlusion is set for each eye on liquid crystal glasses that are worn for 6-8 hours a day during 6 to 12 months.

The present method relates to the field of ophthalmology and is intended for postoperative restoring bifoveal fusion in patients, inter alia in children, following concomitant esotropia surgery, with use of liquid crystal (LC) glasses.

Liquid crystal glasses are used in ophthalmology to treat amblyopia and restore binocular and stereoscopic vision.

A method to test and restore binocular vision with the use of LC glasses (RU patent No. 2133103) is known. The method includes presenting images to each eye alternately at a switching frequency of 50 Hz which is digitally synchronized with the presentation of a visual signal on a video monitor screen to a corresponding eye. This has beneficial effects when the fusion reflex is normal.

The disadvantage of this method is inability to use it for restoring a sensory fusion due to the high switching frequency and inability to take into account the duration of fixative eye movements required for personalized programming of glasses as well as for adequately presenting visual information to the right and left eyes. Furthermore this method may not be used for infants (2 to 7 years old).

The closest analogous solution is the method for restoring bifoveal fusion, whereby restoration of the sensory fusion in patients with a residual angle of deviation of less than 10 degrees after concomitant esotropia surgery is performed after determination of the duration (t, ms) of patient's fixative eye movement by means of video-oculography tracking device followed by calculation of alternation frequency ν in Hz using the formula:

ν=500/(t+40), where
t is the duration (ms) of the fixative eye movement,
40 is the minimum time (ms) required for visualization between alternations (RU patent No. 2721888).

The disadvantage of this method is missing calculations of the duration of occlusion for each single eye as a function of a velocity difference between the refixation movements.

An object of the invention is to provide an effective method for restoring bifoveal fusion.

The technical effect of the invention is restoration of bifoveal fusion in accordance with individual ocular motility parameters, using anharmonical alternating of liquid crystal glasses.

The technical effect is achieved by the approach, during which the duration (t, ms) of the fixative eye movement is determined for each eye with the Gazelab video eye tracking device, by BCN Innova (Spain), in patients having a residual angle of deviation of less than 10 degrees after concomitant esotropia surgery followed by calculation of duration of occlusion in ms for each eye, by the formula:

t=40+U, where
t is the duration of occlusion (ms);
40 is the minimum time (ms) required for visualization between alternations;
U is the duration of the fixative eye movement (ms),
and the duration of occlusion is set for each eye on liquid crystal glasses that are to be worn for 6 to 8 hours a day during 6 to 12 months.

Example 1. Patient K., 4 years old, after surgical treatment of congenital non-accommodative esotropia. The postsurgical angle of deviation was 4 degrees. The visual acuity in both the right and left eyes was 1.0. The postsurgical examination by a synoptophore has diagnosed absence of sensory fusion for the patient. The vision character assessed by the Worth 4-dot test at 5 meters was simultaneous. By video-oculography it has been found that the fixative eye movements of the right and left eye have the durations of 180 ms and 220 ms, respectively. Using the formula, the required duration of occlusion has been calculated at 220 and 260 ms for the right-eye and left-eye lenses of liquid crystal glasses, respectively. This duration has been set on the liquid crystal glasses. Wearing the liquid crystal glasses for 6 hours a day during 6 months has provided successful restoration of the sensory fusion as well as, by further diploptic treatment, of the binocular vision.

Example 2. Patient L., 22 years old, after surgical treatment of non-accommodative esotropia. The angle of deviation was 8 degrees. The visual acuity was 0.8 in the right eye after correction and 1.0 in the left eye. The postsurgical examination by a synoptophore has diagnosed absence of sensory fusion of the patient. The vision character assessed by the Worth 4-dot test at 5 meters was simultaneous. By video-oculography it has been found that the refixation movements of the right and left eyes have the durations of 160 ms and 230 ms, respectively. Using the formula, the required duration of occlusion has been calculated at 200 and 270 ms for the right-eye and left-eye lenses of liquid crystal glasses, respectively. This duration has been set on the liquid crystal glasses. Wearing the liquid crystal glasses for 8 hours a day during 12 months has provided successful restoration of the sensory fusion.

Example 3. Patient M., 3 years old, after surgical treatment of congenital non-accommodative esotropia. The postsurgical angle of deviation was 6 degrees. The visual acuity in both the right and left eyes was 0.8-1.0. The postsurgical examination by a synoptophore has diagnosed absence of sensory fusion of the patient. The vision character assessed by the Worth 4-dot test at 5 meters was simultaneous. By video-oculography it has been found that the refixation movements of the right and left eyes have the durations of 160 ms and 180 ms, respectively. Using the formula, the required duration of occlusion has been calculated at 200 and 220 ms for the right-eye and left-eye lenses of liquid crystal glasses, respectively. This duration has been set on the liquid crystal glasses. Wearing the liquid crystal glasses for 6 hours a day during 9 months has provided successful restoration of the sensory fusion as well as, after further diploptic treatment, of the binocular vision.

1. A method for restoring bifoveal fusion in the case of anisomotility of patients with a residual angle of deviation less than 10 degrees after esotropia surgery, the method comprising determining a duration of the refixation eyeball movement with the use of a video eye tracking device, characterized in that the duration of the fixative eye movement is determined for each eye in ms, a duration of occlusion is then calculated in ms for each eye, by the formula:
t=40+U, where
t is the duration of occlusion (ms);
40 is the minimum time (ms) required for visualization between alternations;
U is the duration of the fixative eye movement (ms),
and the duration of occlusion is set for each eye on liquid crystal glasses that are worn for 6 to 8 hours a day during 6 to 12 months.
